(19) United States
(12) Patent Application Publication
(10) Pub. No.: US 2021/0161114 A1
(43) Pub. Date: Jun. 3, 2021

(54) Vip3A RESISTANT SPODOPTERA FRUGIPERDA

(71) Applicant: SYNGENTA PARTICIPATIONS AG, Basel (CH)

(72) Inventors: Julio Fatoretto, San Paulo (BR); Zhimou Wen, Research Triangle Park, NC (US); Phillip Matthews, Research Triangle Park, NC (US); Jeng Shong Chen, Research Triangle Park, NC (US)

(73) Assignee: SYNGENTA PARTICIPATIONS AG, Basel (CH)

(21) Appl. No.: 17/173,563

(22) Filed: Feb. 11, 2021

Related U.S. Application Data
(62) Division of application No. 15/770,764, filed on Apr. 25, 2018.
(60) Provisional application No. 62/257,922, filed on Nov. 20, 2015.

(51) Int. Cl. A01K 67/033; C07K 14/325; A01N 63/00; C12N 15/82; A01N 63/10; A01N 37/46

(52) U.S. Cl. CPC A01K 67/033; C07K 14/325; A01N 63/00; A01K 2227/706; A01N 63/10; A01N 37/46; Y02A 40/146; C12N 15/8286

(57) ABSTRACT

Artificially selected strains of insects from the genus Spodoptera exhibiting resistance to a Bacillus thuringiensis derived Vip3A protein are described. Methods for various uses of these strains are also described.

### RELATED APPLICATIONS

This application is a divisional of U.S. patent application Ser. No. 15/770,764 filed on Apr. 25, 2018, which is a national stage application of International Application No. PCT/US2016/031958 filed on May 12, 2016, which claims the benefit of U.S. Provisional Application No. 62/257,922 filed on Nov. 20, 2015, the entire contents of each of which are incorporated by reference herein.

### FIELD OF THE INVENTION

The present invention relates generally to the control of pests that cause damage to crop plants. More specifically, the present invention relates to artificially selected strains of Spodoptera frugiperda resistant to a Bacillus thuringiensis-derived protein Vip3A. Methods for various uses of these strains are also described.

### BACKGROUND

Plant pests are a major factor in the loss of the world's important agricultural crops. About $8 billion are lost every year in the U.S. alone due to infestations of non-mammalian pests including insects. Insect pests are mainly controlled by intensive applications of chemical pesticides, which are active through inhibition of insect growth, prevention of insect feeding or reproduction, or cause death. Good insect control can thus be reached, but these chemicals can sometimes also affect other, beneficial insects. Another problem resulting from the wide use of chemical pesticides is the appearance of resistant insect varieties. This has been partially alleviated by various resistance management practices, but there is an increasing need for alternative pest control agents. Biological pest control agents, such as Bacillus thuringiensis strains expressing pesticidal toxins like δ-endotoxins, have also been applied to crop plants with satisfactory results, offering an alternative or complement to chemical pesticides. In particular, the expression of insecticidal toxins in transgenic plants, such as B. thuringiensis δ-endotoxins, has provided efficient protection against selected insect pests, and transgenic plants expressing such toxins have been commercialized, allowing farmers to reduce applications of chemical insect control agents.

Another family of insecticidal proteins produced by Bacillus species during the vegetative stage of growth (vegetative insecticidal proteins (Vip)) has also been identified. U.S. Pat. Nos. 5,877,012, 6,107,279, and 6,137,033, herein incorporated by reference, describe a class of insecticidal proteins called Vip3. Other disclosures, including WO 98/18932, WO 98/33991, WO 98/00546, and WO 99/57282, have also now identified homologues of the Vip3 class of proteins. Vip3 coding sequences encode approximately 88 kDa proteins that possess insecticidal activity against a wide spectrum of lepidopteran pests, including but not limited to black cutworm (BCW, Agrotis ipsilon), fall armyworm (FAW, Spodoptera frugiperda), tobacco budworm (TBW, Heliothis virescens), sugarcane borer (SCB, Diatraea saccharalis), lesser cornstalk borer (LCB, Elasmopalpus lignosellus), and corn earworm (CEW, Helicoverpa zea). When expressed in transgenic plants, for example maize (Zea mays), Vip3 coding sequences confer protection to the plant from insect feeding damage.

Fall armyworm is one of the main target pests of Vip3A transgenic maize (U.S. Pat. Nos. 6,107,279, 8,232,456, 8,232,456, 8,455,720, and 8,618,272, herein incorporated by reference). This species is considered the most destructive pest of corn because of the high reproductive capacity and adult dispersion, overlapping generations throughout the year, and the availability of host crops in different agro-ecosystems. Evolution of resistance to Cry1F corn in S. frugiperda was documented in less than four years from commercial release in Brazil (Farias et al., 2014, Crop Protection, 64: 150-158). Field-evolved resistance to Cry1F in fall armyworm was also reported in Puerto Rico (Storer et al., 2010, J Econ Entomol, 103: 1031-1038), and recently Cry1F resistant FAW has been found in the southern United States (Huang et al., 2014, PLoS ONE, 11: e112958).

In this context, the Vip insecticidal proteins offer a promising alternative for resistance management of S. frugiperda. Vip proteins present a different mode of action from Cry proteins. Vip proteins are exotoxins produced and secreted during the vegetative growing stage of B. thuringiensis, while Cry proteins are produced at the sporulation stage. Moreover, Vip proteins have distinct binding properties and lack sequence homology with any Cry proteins (Lee et al., 2003, Appl Environ Microbiol, 69: 4648-4657). These differences cause pore formation with unique properties, indicating a low potential for cross-resistance (Gouffon et al., 2011, Appl Environ Microbiol, 77: 3182-3188). Therefore, Vip proteins may be efficient in the control of the evolution of insects resistant to Cry proteins (e.g. Cry1F).

The establishment of insect resistance management strategies is essential to extend the lifetime of Bt proteins. For resistance management, the refuge plus high-dose strategy is central to the resistance management of many transgenic crops producing Bt proteins. This strategy is based on three components. First, the transgenic plant tissue should be highly toxic to a target pest, so that insect resistance is functionally recessive and insects that are heterozygous for resistance are susceptible to the Bt protein. Second, resistance alleles should be rare, so that there would be few homozygous survivors. Finally, the transgenic crop should be planted with nontoxic refuges interspersed, so that resistant homozygotes will mate randomly or preferentially with susceptible homozygotes, thereby producing heterozygous progeny that is susceptible to the transgenic plant tissue. This strategy is currently recommended and used to prevent or delay resistance evolution to Bt crops. However, before resistance evolution occurs in the field, the assumptions and predictions of these resistance management strategies, such as the genetic characterization of resistance, need to be tested. This can only be accomplished by the artificial selection of resistant strains, which are selected through human intervention. Artificially selected resistant strains offer an opportunity to assess resistance inheritance, determine the mechanism of resistance, estimate the resistance allele frequency, evaluate the presence of fitness cost, develop molecular diagnostics, test mathematical models to predict resistance evolution, and to refine the resistance management strategies in use.

### SUMMARY OF THE INVENTION

The present invention provides an artificially selected insect from the genus Spodoptera comprising resistance to a Vip3A protein when compared to an insect not selected for resistance to a Vip3A protein, or an insect that is susceptible to a Vip3A protein. The Vip3A protein may be a Vip3Aa, a Vip3Aa19, and/or a Vip3Aa20 protein. In other embodiments, the Vip3A protein may be Vip3Aa20 protein from a MIR162 transgenic corn cell.

The present invention provides an artificially selected insect from the genus Spodoptera comprising resistance to a Vip3A protein, where the artificially selected insect is from the species Spodoptera frugiperda (fall armyworm). The Vip3A resistance is conferred by a genetically inherited trait or traits. In some embodiments, a fitness cost is associated with the resistance to a Vip3A protein.

In some embodiments, the artificially selected insect from the genus Spodoptera comprising resistance to a Vip3A protein is derived from an insect collected from North America or South America. In some embodiments, the artificially selected insect is derived from an insect collected from the United States of America or Brazil. In some embodiments, the artificially selected insect is derived from an insect collected from Georgia, the United States of America, or Bahia, Brazil. In some embodiments, the artificially selected insect is derived from an insect collected from Tifton, Ga., the United States of America, or Correntina, Bahia, Brazil.

The invention further provides a method of evaluating the activity of a compound on an artificially selected fall armyworm comprising resistance to a Vip3A protein compared to an insect not selected for resistance to a Vip3A protein, or an insect that is susceptible to a Vip3A protein. This method involves exposing a group of Vip3A-resistant fall armyworms to a compound, wherein said group of fall armyworms comprises resistance to a Vip3A protein; and evaluating the activity of the compound on the group of one or more fall armyworms to determine if the compound is toxic to a Vip3A-resistant fall armyworm. In some embodiments, the method further comprises selecting a compound for further development when said compound exhibits toxicity to a Vip3A-resistant fall armyworm. The present invention further provides a compound selected according to the method.

The invention is further drawn to a method for producing a field-derived, artificially selected strain of fall armyworms that comprises resistance to a Vip3A protein. This method involves collecting fall armyworms from a geographic location, where a geographic location refers to a position on planet Earth. Examples of a geographic location include a grassland, a prairie, an unused field, a fallow field, an agricultural field, an area proximal to an agricultural field, a hedgerow, an agricultural field where corn is grown or an agricultural field comprising maize plants that express Vip3A, such as MIR162 transgenic maize plants. The collected fall armyworms are then maintained in an artificial environment, such as in a plastic chamber or in a greenhouse with a screen to keep them physically isolated from the rest of the environment, and allowed to feed on a diet comprising an effective concentration of Vip3A, wherein the effective concentration is sufficient to kill susceptible fall armyworms. This diet may be an artificial diet that is supplemented with Vip3A protein at an effective concentration, or it may be tissue from MIR162 transgenic maize plants. The surviving fall armyworms are then selected. In some embodiments, the zygosity of the Vip3A resistance trait may then be characterized. A colony is then formed with the surviving fall armyworms that comprise resistance to Vip3A. The zygosity may be determined by crossing survivors of the F2 generation and determining the Vip3A resistance of the subsequent generation. This process may be repeated for subsequent generations as necessary, until a colony or strain is identified in which all the fall armyworms are Vip3A-resistant. This method will create a strain of fall armyworms which comprises resistance to Vip3A and preferably is homozygous for the Vip3A-resistance trait. In a further embodiment, Vip3A-resistant fall armyworms from the strain created above are mated with fall armyworms that are susceptible to Vip3A, thereby producing progeny which are then feed on a diet comprising an effective concentration of Vip3A, wherein the effective concentration is sufficient to kill susceptible fall armyworms. The number of surviving fall armyworms from each initial mating is counted and the mortality rate is determined and analyzed. In some embodiments, the surviving Vip3A-resistant fall armyworms may then be further backcrossed to the Vip3A-resistant fall armyworms and the mortality rate of the subsequent progeny on a diet comprising Vip3A determined. This method is useful for determining the genetic inheritance and genetic stability of the Vip3A-resistant trait.

The invention is further drawn to a method for producing an artificially selected strain of Vip3A-resistant fall armyworms. This method involves collecting fall armyworms from a geographic location, where a geographic location refers to a position on planet Earth. Examples of a geographic location include a grassland, a prairie, an unused field, a fallow field, an agricultural field, an area proximal to an agricultural field, a hedgerow, or an agricultural field where corn is grown. The initially collected fall armyworms are allowed to breed unselectively for one generation, thereby producing an F1 generation which has not been feed a diet comprising a Vip3A protein. The sex of the F1 adults is determined and breeding pairs are selected for producing the F2 generation. The larvae of the F2 generation are then allowed to feed on a diet comprising an effective concentration of Vip3A. The surviving fall armyworms are then selected. In some embodiments, the zygosity of the Vip3A resistance trait may then be characterized. The zygosity may be determined by crossing survivors of the F2 generation and determining the Vip3A resistance of the subsequent generation. This process may be repeated for subsequent generations as necessary, until a strain is identified in which all the fall armyworms are Vip3A-resistant. This method will create a strain of fall armyworms which comprises resistance to Vip3A and preferably is homozygous for the Vip3A-resistance trait. In a further embodiment, Vip3A-resistant fall armyworms from the strain created above are mated with fall armyworms that are susceptible to Vip3A, thereby producing progeny which are then feed on a diet comprising an effective concentration of Vip3A. The number of surviving fall armyworms from each initial mating is counted and the mortality rate is determined and analyzed. In some embodiments, the surviving Vip3A-resistant fall armyworms may then be further backcrossed to the Vip3A-resistant fall armyworms and the mortality rate of the subsequent progeny on a diet comprising Vip3A determined. This method is useful for determining the genetic inheritance and genetic stability of the Vip3A-resistant trait.

### DETAILED DESCRIPTION OF THE INVENTION

It is to be understood that this invention is not limited to the particular methodology, protocols, cell lines, plant species or genera, constructs, and reagents described herein as such. It is also to be understood that the terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a plant” is a reference to one or more plants and includes equivalents thereof known to those skilled in the art, and so forth. As used herein, the word “or” means any one member of a particular list and also includes any combination of members of that list (i.e., includes also “and”).

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). With regard to a temperature the term “about” means±1° C., preferably ±0.5° C. Where the term “about” is used in the context of this invention (e.g., in combinations with temperature or molecular weight values) the exact value (i.e., without “about”) is preferred.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The term “insect” as used herein includes any organism now known or later identified that is classified in the animal kingdom, phylum Arthropoda, class Insecta, including but not limited to insects in the orders Coleoptera (beetles), Lepidoptera (moths, butterflies), Diptera (flies), Protura, Collembola (springtails), Diplura, Microcoryphia (jumping bristletails), Thysanura (bristletails, silverfish), Ephemeroptera (mayflies), Odonata (dragonflies, damselflies), Orthoptera (grasshoppers, crickets, katydids), Phasmatodea (walkingsticks), Grylloblattodea (rock crawlers), Mantophasmatodea, Dermaptera (earwigs), Plecoptera (stoneflies), Embioptera (web spinners), Zoraptera, Isoptera (termites), Mantodea (mantids), Blattodea (cockroaches), Hemiptera (true bugs, cicadas, leafhoppers, aphids, scales), Thysanoptera (thrips), Psocoptera (book and bark lice), Phthiraptera (lice; including but not limited to suborders Amblycera, Ischnocera and Anoplura), Neuroptera (lacewings, owlflies, mantispids, antlions), Hymenoptera (bees, ants, wasps), Trichoptera (caddisflies), Siphonaptera (fleas), Mecoptera (scorpion flies), Strepsiptera (twisted-winged parasites), and any combination thereof.

Insects in the order Lepidoptera include without limitation any insect now known or later identified that is classified as a lepidopteran, including those insect species within suborders Zeugloptera, Glossata, and Heterobathmiina, and any combination thereof. Exemplary lepidopteran insects include, but are not limited to, Ostrinia spp. such as O. nubilalis (European corn borer); Plutella spp. such as P. xylostella (diamondback moth); Spodoptera spp. such as S. frugiperda (fall armyworm), S. ornithogalli (yellowstriped armyworm), S. praefica (western yellowstriped armyworm), S. eridania (southern armyworm) and S. exigua (beet armyworm); Agrotis spp. such as A. ipsilon (black cutworm), A. segetum (common cutworm), A. gladiaria (claybacked cutworm), and A. orthogonia (pale western cutworm); Striacosta spp. such as S. albicosta (western bean cutworm); Helicoverpa spp. such as H. zea (corn earworm), H. punctigera (native budworm), S. littoralis (Egyptian cotton leafworm) and H. armigera (cotton bollworm); Heliothis spp. such as H. virescens (tobacco budworm); Diatraea spp. such as D. grandiosella (southwestern corn borer) and D. saccharalis (sugarcane borer); Trichoplusia spp. such as T. ni (cabbage looper); Sesamia spp. such as S. nonagroides (Mediterranean corn borer); Pectinophora spp. such as P. gossypiella (pink bollworm); Cochylis spp. such as C. hospes (banded sunflower moth); Manduca spp. such as M. sexta (tobacco hornworm) and M. quinquemaculata (tomato hornworm); Elasmopalpus spp. such as E. lignosellus (lesser cornstalk borer); Pseudoplusia spp. such as P. includens (soybean looper); Anticarsia spp. such as A. gemmatalis (velvetbean caterpillar); Plathypena spp. such as P. scabra (green cloverworm); Pieris spp. such as P. brassicae (cabbage butterfly), Papaipema spp. such as P. nebris (stalk borer); Pseudaletia spp. such as P. unipuncta (common armyworm); Peridroma spp. such as P. saucia (variegated cutworm); Keiferia spp. such as K. lycopersicella (tomato pinworm); Artogeia spp. such as A. rapae (imported cabbageworm); Phthorimaea spp. such as P. operculella (potato tuberworm); Crymodes spp. such as C. devastator (glassy cutworm); Feltia spp. such as F. ducens (dingy cutworm); and any combination of the foregoing.

The terms “pest” and “plant pest” are used interchangeably herein. These terms include, but are not limited to, insect pests, nematode pests and mite pests.

As used herein, a “colony” refers to several insects, all of the same species, which live together in close association. A “strain” is a group of insects, all of the same species, that have some known characteristic that differentiates them from other insects of the same species. In some embodiments, this characteristic is genetically inherited. In some embodiments, this characteristic is a trait, for example resistance to a pesticide.

As used herein, “resistance”, “resistant”, or “resistant-” refers to a genetically based decrease in susceptibility to a pesticide. The pesticide described in the present application is an insecticide, in preferred embodiments an insecticidal protein, in more preferred embodiments a Vip protein derived from B. thuringiensis, in more preferred embodiments a Vip3 protein, in more preferred embodiments a Vip3A protein, in more preferred embodiments a Vip3Aa protein, in more preferred embodiments a Vip3Aa19 or a Vip3Aa20 protein. The term “Vip3A-resistant” refers to an insect with resistance to the insecticidal protein Vip3A. A Vip3A, Vip3Aa, Vip3Aa19, or Vip3Aa20 protein may be provided in the insect diet as a purified supplement. Alternatively, a Vip3Aa20 protein may be provided in the diet as a protein expressed in a transgenic plant, where tissues from the transgenic plant are provided as the insect diet. Agrisure® Viptera® and Agrisure® Viptera® 3 corn are commercially available corn varieties which comprise the transgenic event MIR162. MIR162 expresses Vip3Aa20 protein (U.S. Pat. Nos. 8,455,720, 8,618,272, and 8,232,456, incorporated by reference herein).

As used herein, a “resistant strain” or “resistant individual” refers to a strain or individual with a genetically based decrease in susceptibility to a pesticide relative to other individuals of the same species.

As used herein, “susceptibility” or “sensitivity” refers to the tendency to be killed or harmed by a pesticide.

By “activity” of a Vip insecticidal protein is meant that the Vip protein functions as an orally active insect control agent, has a toxic effect, or is able to disrupt or deter insect feeding, which may or may not cause death of the insect. When a Vip protein is delivered to the insect, either as a bacterially-produced protein supplemented in a diet or transgenically expressed in provided plant tissues, the result is typically death of the insect, or the insect does not feed upon the source that makes the Vip protein available to the insect. As used herein, “toxicity” refers to the decreased viability of a cell, and “viability” refers to the ability of a cell to proliferate and/or differentiate and/or maintain its biological characteristics in a manner characteristic of that cell in the absence of a toxic compound.

As used herein, the term “control” of pest infestation refers to any effect on a pest that serves to limit and/or reduce either the numbers of pest organisms and/or the damage caused by the pest. To “control” pests may or may not mean killing the pests, although it preferably means killing the pests.

As used herein the term transgenic “event” refers to a recombinant plant produced by transformation and regeneration of a plant cell or tissue with heterologous DNA, for example, an expression cassette that includes a gene of interest. The term “event” refers to the original transformant and/or progeny of the transformant that include the heterologous DNA. The term “event” also refers to progeny produced by a sexual outcross between the transformant and another corn line. Even after repeated backcrossing to a recurrent parent, the inserted DNA and the flanking DNA from the transformed parent is present in the progeny of the cross at the same chromosomal location. The term “event” also refers to DNA from the original transformant comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA. Normally, transformation of plant tissue produces multiple events, each of which represent insertion of a DNA construct into a different location in the genome of a plant cell. Based on the expression of the transgene or other desirable characteristics, a particular event is selected. Thus, “event XXX”, “XXX” or “XXX event” may be used interchangeably.

The term “bioassay” refers to a test in which a group of live organisms is exposed to a pesticide, for example an insecticide, a toxin, or an insecticidal protein, to evaluate the susceptibility of the organism to the pesticide.

As used herein, the “EC₅₀”, or median effective concentration, is the concentration of pesticide that causes a specific response, such as for example the failure to emerge as an adult, in 50% of the individuals in a population. The “IC₅₀”, or median inhibitory concentration, is the concentration of a pesticide that inhibits an essential process such as growth or feeding in 50% of the individuals in a population. The “LC₅₀”, or median lethal concentration, is the concentration of a pesticide that kills 50% of the individuals in a population. The “LD₅₀”, or median lethal dose, is the dose of a pesticide that kills 50% of the individuals in a population.

“Effective insect-controlling amount” means that concentration of toxin that inhibits, through a toxic effect, the ability of insects to survive, grow, feed and/or reproduce, or to limit insect-related damage or loss in crop plants. “Effective insect-controlling amount” may or may not mean killing the insects, although it preferably means killing the insects.

As used herein, the term “resistance management” refers to tactics implemented to delay evolution of resistance in pest populations. “Resistance monitoring” refers to systematic testing of organisms with bioassays, biochemical tests (e.g., enzyme assays), or molecular tests (e.g., DNA screening) to assess the frequency, magnitude, and spatial pattern of resistance.

As used herein, the term “resistance ratio” refers to an index of the magnitude of resistance often calculated as the LC₅₀ for a resistant population divided by the LC₅₀ for a susceptible population; it can also be calculated analogously for other parameters that specify the amount of pesticide that causes a response in a specified percentage of a population such as LC₅₀, LC₉₅, LD₅₀, LD₉₅, EC₅₀, EC₉₅, IC₅₀, or IC₉₅.

As used herein, “artificial selection” is performed by the hand of man to produce an organism, strain, or colony which comprises a desired trait. An “artificially selected” insect, strain, or colony is one in which the hand of man has provided a selection pressure to produce the artificially selected insect, strain, or colony which comprises a desired trait. For example, an artificially selected insect strain may comprise resistance to a pesticide as a result of artificial selection using said pesticide. The first step of artificial selection is to provide a selection pressure, such as an effective insect-controlling amount of a pesticide on a population of insects. In some embodiments, the source of selection pressure, such as the pesticide, is removed after a period of time to allow the survivors to complete their lifecycles in the absence of the selection pressure. Subsequently, the surviving insects are selected; optionally the zygosity of the surviving insects is determined. The surviving insects may be allowed to breed for at least one generation to generate more insects, a colony, or a strain comprising the desired trait, for example resistance to the pesticide. In some embodiments, artificial selection is performed on at least one subsequent generation.

Artificial selection can be performed in a laboratory setting, for example in a bioassay where the insects are maintained in vitro (such as in a plastic chamber or well or multitude of wells or chambers) and are fed, for example, artificial diet comprising a pesticide where the pesticide may be a Bt insecticidal protein, a Vip protein, a Vip3A protein, a Vip3Aa protein, a Vip3Aa19 protein, and/or a Vip3Aa20 protein. Insects may also be fed an artificial diet comprising pieces of transgenic plant tissue for example plant tissue derived from transgenic corn event MIR162. Following an appropriate length of time where the susceptible insects are likely to have consumed an effective insect-controlling amount, the remaining insects may be collected and may be placed on a diet which does not comprise the insecticidal agent. Alternatively, the insects may be maintained on a diet comprising the pesticide for their entire life-cycle. The surviving insects may be allowed to breed for at least one generation. The progeny may then undergo at least one round of the same or similar artificial selection to produce an artificially selected insect strain that comprises resistance to said insecticidal agent.

Artificial selection may also be performed in a greenhouse-type setting, where for example insects are placed on intact transgenic plants expressing an insecticidal agent, where said plants are growing in a greenhouse, growth chamber, or otherwise in an indoor setting such as within a laboratory, and where the insecticidal agent may be an insecticidal protein, a Vip protein, a Vip3A protein, a Vip3Aa protein, a Vip3Aa19 protein, and/or a Vip3Aa20 protein. Following an appropriate length of time where the susceptible insects are likely to have consumed an effective insect-controlling amount, the remaining insects may be collected and may be placed on a diet which does not comprise the insecticidal agent. Alternatively, the insects may be maintained on a diet comprising the pesticide for their entire life-cycle. The surviving insects may be allowed to breed for at least one generation. The progeny may then undergo the same or a similar round of artificial selection to produce an artificially selected insect strain that comprises resistance to said insecticidal agent.

Artificial selection may also be performed in a field setting, where for example insects are placed on intact transgenic plants expressing an insecticidal agent, where said plants are growing in a field, and where the insecticidal agent may be an insecticidal protein, a Vip protein, a Vip3A protein, and/or a Vip3Aa20 protein. The field or the plants in the field may have a physical barrier, for example netting, which would prevent insects from escaping or entering into the field or onto the plants. Following an appropriate length of time where the susceptible insects are likely to have consumed an effective insect-controlling amount, the remaining insects would be collected. Following an appropriate length of time where the susceptible insects are likely to have consumed an effective insect-controlling amount, the remaining insects may be collected and may be placed on a diet which does not comprise the insecticidal agent. Alternatively, the insects may be maintained on a diet comprising the pesticide for their entire life-cycle. The surviving insects would be allowed to breed for at least one generation. The progeny may then undergo at least one round of the same or similar artificial selection to produce an artificially selected insect strain that comprises resistance to said insecticidal agent.

An artificially selected insect or a laboratory-reared insect is derived from an insect collected from a geographic location, as the insect cannot be synthesized. The derivation may be from a parental insect over 1 generation prior to the present artificially selected insect, over 2 generations prior, over 3 generations prior, over 5 generations prior, over 7 generations prior, over 10 generations prior, over 20 generations prior, over 30 generations prior, over 40 generations prior, over 50 generations prior, over 60 generations prior, over 70 generations prior, over 80 generations prior, over 90 generations prior, over 100 generations prior, over 1,000 generations prior, over 1 million generations prior, over 1 billion generations prior, or over 1 trillion generations prior to the present artificially selected insect.

“Field-evolved resistance” or “field-selected resistance” is a genetically based decrease in susceptibility of a population to a pesticide caused by exposure to the pesticide in the field. “Practical resistance” is field-selected resistance that reduces the efficacy of a pesticide and has practical consequences for pest control (Tabashnik et al., 2014, J of Economic Entomology, 107: 496-507; Zhang et al., 2012, PNAS, 109: 10275-10280).

Fitness costs have been found in some insect strains resistant to pesticides. An inherited trait is associated with a fitness cost when alleles conferring higher fitness in the presence of a pesticide reduce fitness in the absence of the pesticide. In the absence of the pesticide, fitness may be lower for resistant populations than for susceptible insects. In some cases, artificially selected resistant insects may have reduced fitness when being fed on plants that are the normal host for the insect. The fitness cost may be linked to development time and survival rate of egg, larvae, pupae and egg to adult period; emergence rates, sex ratio; female longevity; timing of pre-oviposition, oviposition and post-oviposition, and/or fecundity (total eggs per female). While fitness costs can cause a challenge for resistance selection, they can be valuable tools for resistance management and Bt protein product longevity.

A “gene” is defined herein as a hereditary unit consisting of a polynucleotide that occupies a specific location on a chromosome and that contains the genetic instruction for a particular characteristic or trait in an organism, or such hereditary unit from a group of heterologous organisms depending on context.

The term “genotype” refers to the genetic constitution of a cell or organism. An individual's “genotype for a set of genetic markers” includes the specific alleles, for one or more genetic marker loci, present in the individual. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest (e.g., a quantitative trait as defined herein). Thus, in some embodiments a genotype comprises a sum of one or more alleles present within an individual at one or more genetic loci of a quantitative trait.

The term “locus” refers to a position (e.g., of a gene, a genetic marker, or the like) on a chromosome of a given species.

“PCR (polymerase chain reaction)” is understood within the scope of the invention to refer to a method of producing relatively large amounts of specific regions of DNA, thereby making possible various analyses that are based on those regions.

“Polymorphism” is understood within the scope of the invention to refer to the presence in a population of two or more different forms of a gene, genetic marker, or inherited trait.

The term “allele(s)” means any of one or more alternative forms of a gene, wherein all alleles relate to at least one trait or characteristic. In a diploid cell, the two alleles of a given gene occupy corresponding loci on a pair of homologous chromosomes. In some instances (e.g., for QTLs) it is more accurate to refer to “haplotype” (i.e., an allele of a chromosomal segment) instead of “allele”, however, in those instances, the term “allele” should be understood to comprise the term “haplotype”. If two individuals possess the same allele at a particular locus, the alleles are termed “identical by descent” if the alleles were inherited from one common ancestor (i.e., the alleles are copies of the same parental allele). The alternative is that the alleles are “identical by state” (i.e., the alleles appear to be the same but are derived from two different copies of the allele). Identity by descent information is useful for linkage studies; both identity by descent and identity by state information can be used in association studies, although identity by descent information can be particularly useful.

The term “backcrossing” is understood within the scope of the invention to refer to a process in which a hybrid progeny is crossed back to one of the parents at least one time.

The phrase “phenotypic trait” refers to the appearance or other detectable characteristic of an individual, resulting from the interaction of its genome with the environment.

The term “plurality” refers to more than one entity. Thus, a “plurality of individuals” refers to at least two individuals. In some embodiments, the term plurality refers to more than half of the whole. For example, in some embodiments a “plurality of a population” refers to more than half the members of that population.

The term “progeny” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

The present invention describes two separate Fall armyworm (S. frugiperda) artificially selected strains which have resistance to a Vip3A protein. Fall armyworms are known to be a pest of corn, and are responsible for significant negative economic impact in corn agro-business. Additionally, fall armyworms have been shown to rapidly evolve resistance to insecticidal proteins expressed in transgenic maize (Farias et al., 2014, Crop Protection 64: 150-158). The artificially selected strains of the present invention have value because they provide opportunities to, for example, assess resistance inheritance, determine the mechanism of resistance, evaluate the presence of fitness cost, develop molecular diagnostics, and to refine the resistance management strategies in use. Despite the value of these strains, no Vip3A-resistant strains have been previously documented to our knowledge. Therefore, the artificial selection and creation of these strains fill an unmet need.

The life cycle of fall armyworms comprises an egg stage, followed by a larva stage that typically comprises six instars. A “neonate” is a recently hatched, first instar larva, which has not yet eaten a meal. Larvae cause damage by consuming foliage. Young larvae initially consume leaf tissue from one side, leaving the opposite epidermal layer intact. By the second or third instar, larvae begin to make holes in leaves, and eat from the edge of the leaves inward. Feeding in the whorl of corn often produces a characteristic row of perforations in the leaves. Larval densities are usually reduced to one to two per plant when larvae feed in close proximity to one another, due to cannibalistic behavior. In corn, they sometimes burrow into the ear, feeding on kernels in the same manner as corn earworm, Helicoverpa zea. Unlike corn earworm, which tends to feed down through the silk before attacking the kernels at the tip of the ear, fall armyworm will feed by burrowing through the husk on the side of the ear.

Pupation normally takes place in the soil. The resulting moths are nocturnal, and are most active during warm, humid evenings. After a preoviposition period of three to four days, the female normally deposits most of her eggs during the first four to five days of life. Duration of adult life is estimated to range from about seven to 21 days.

The present invention provides an artificially selected insect from the genus Spodoptera comprising resistance to a Vip3A protein when compared to an insect not selected for resistance to a Vip3A protein, or an insect that is susceptible to a Vip3A protein. The Vip3A protein may be a Vip3Aa protein, a Vip3Aa19 protein, and/or a Vip3Aa20 protein. In some embodiments, the Vip3A protein may be Vip3Aa20 protein from a MIR162 transgenic corn cell.

The present invention provides an artificially selected insect from the genus Spodoptera comprising resistance to a Vip3A protein, where the artificially selected insect is from the species Spodoptera frugiperda (fall armyworm). In some embodiments, the resistance is conferred by an autosomal recessive trait. In some embodiments, the resistance is conferred by a sex-linked trait. In some embodiments, the resistance is conferred by an autosomal dominant, co-dominant, and/or functionally dominant trait. In some embodiments, a fitness cost is associated with the resistance to a Vip3A protein. In some embodiments, the fitness cost may be linked to development time and survival rate of egg, larvae, pupae and egg to adult period; emergence rates, sex ratio; female longevity; timing of pre-oviposition, oviposition and post-oviposition, and/or fecundity (total eggs per female).

In some embodiments, the artificially selected insect from the genus Spodoptera comprising resistance to a Vip3A protein is derived from an insect collected from North America or South America. In some embodiments, the artificially selected insect is derived from an insect collected from the United States of America or Brazil. In some embodiments, the artificially selected insect is derived from an insect collected from Georgia, the United States of America, or Bahia, Brazil. In some embodiments, the artificially selected insect is derived from an insect collected from Tifton, Ga., the United States of America, or Correntina, Bahia, Brazil.

The invention further provides a method of evaluating the activity of a compound on an artificially selected fall armyworm comprising resistance to a Vip3A protein compared to an insect not selected for resistance to a Vip3A protein, or an insect that is susceptible to a Vip3A protein. This method involves exposing one or more Vip3A-resistant fall armyworms to a compound, wherein said one or more fall armyworms comprises resistance to a Vip3A protein; and evaluating the activity of the compound on the one or more fall armyworms to determine if the compound is toxic to a Vip3A-resistant fall armyworm. Evaluating the activity may comprise measuring the insecticidal activity of the candidate compound. A change in the level of insect mortality or insect vigor in the presence of the compound compared to the level of insect mortality or insect vigor in the absence of the compound indicates that the compound has activity. In some embodiments, the method further comprises selecting a compound for further evaluation when said compound exhibits activity or toxicity to a Vip3A-resistant fall armyworm. Further evaluation may comprise repeating the method more than once, repeating the method on a greater number of Vip3A-resistant fall armyworms, or repeating the method on Vip3A-resistant fall armyworms of the Tifton Vip-R strain and/or of the Correntina Vip-R strain. Further evaluation may also comprise evaluating the activity of the same compound on different insect species, either from the genus Spodoptera or from other insect genera. The present invention further provides a compound selected according to the method.

The invention is further drawn to a method for producing a field-derived, artificially selected strain of fall armyworms that comprises resistance to a Vip3A protein. This method involves collecting a plurality of fall armyworms from a geographic location, where a geographic location refers to a position on planet Earth. Examples of a geographic location include a grassland, a prairie, an unused field, a fallow field, an agricultural field, an area proximal to an agricultural field, a hedgerow, or an agricultural field comprising maize plants. The maize plants in the agriculture field may be transgenic, may express a Bt insecticidal protein, may express a Vip3A protein, or may express a Vip3Aa20 protein, such as MIR162 transgenic maize plants. The collected fall armyworms are then maintained in an artificial environment, such as in a plastic chamber or in a greenhouse with a screen to keep them physically isolated from the rest of the environment, and allowed to feed on a diet comprising an effective concentration of Vip3A, wherein the effective concentration is sufficient to kill susceptible fall armyworms. This diet may be an artificial diet that is supplemented with Vip3A protein at an effective concentration, or it may be tissue from MIR162 transgenic maize plants. The surviving fall armyworms are then selected. In some embodiments, the zygosity of the Vip3A resistance trait may then be characterized. A colony is then formed with the surviving fall armyworms that comprise resistance to Vip3A and preferably are homozyous for the field-evolved Vip3A-resistance trait. In a further embodiment, a Vip3A-resistant fall armyworm from the strain created above is mated with a fall armyworm that is susceptible to Vip3A, thereby producing progeny which are then feed on a diet comprising an effective concentration of Vip3A, wherein the effective concentration is sufficient to kill susceptible fall armyworms. The number of surviving fall armyworms from each initial mating is counted and the mortality rate is determined and analyzed. In some embodiments, the surviving Vip3A-resistant fall armyworms may then be further backcrossed to the Vip3A-resistant fall armyworms and the mortality rate of the subsequent progeny on a diet comprising Vip3A determined. This method is useful for determining the genetic inheritance and genetic stability of the Vip3A-resistant trait.

The invention is further drawn to a method for producing an artificially selected strain of Vip3A-resistant fall armyworms. This method involves collecting a plurality of fall armyworms from a geographic location, where a geographic location refers to a position on planet Earth. Examples of a geographic location include a grassland, a prairie, an unused field, a fallow field, an agricultural field, an area proximal to an agricultural field, a hedgerow, or an agricultural field where corn is grown. The initially collected fall armyworms are considered the F0 generation. They are allowed to breed unselectively for one generation, thereby producing an F1 generation which has not been fed a diet comprising a Vip3A protein. The sex of the F1 adults is determined and breeding pairs are selected for producing the F2 generation. The larvae of the F2 generation are then allowed to feed on a diet comprising an effective concentration of Vip3A, wherein the effective concentration is sufficient to kill susceptible fall armyworms. The surviving fall armyworms are then selected. In some embodiments, the zygosity of the Vip3A resistance trait may then be characterized. The zygosity may be determined by crossing survivors of the F2 generation and determining the Vip3A resistance of the subsequent generation. This process may be repeated for subsequent generations as necessary, until a strain is identified in which all the fall armyworms are Vip3A-resistant. This method will create a strain of fall armyworms which comprises resistance to Vip3A and preferably is homozyous for the Vip3A-resistance trait. In a further embodiment, Vip3A-resistant fall armyworms from the strain created above are mated with fall armyworms that are susceptible to Vip3A, thereby producing progeny which are then fed on a diet comprising an effective concentration of Vip3A, wherein the effective concentration is sufficient to kill susceptible fall armyworms. The number of surviving fall armyworms from each initial mating is counted and the mortality rate is determined and analyzed. In some embodiments, the surviving Vip3A-resistant fall armyworms may then be further backcrossed to the Vip3A-resistant fall armyworms and the mortality rate of the subsequent progeny on a diet comprising Vip3A determined. This method is useful for determining the genetic inheritance and genetic stability of the Vip3A-resistant trait.

For the success of current Vip3A resistance management strategies, it is necessary to continuously monitor the frequency of Vip3A resistance in field populations of fall armyworms, include the presence of a recessive or functionally recessive resistance allele in a heterozygous insect. To do these, there are three primary methods: molecular marker analysis, the F2 screen, and the F1 screen. The F1 screen is more practical and faster than the F2 screen, and most efficient to estimate the frequency of resistance compared to phenotypic monitoring methods that use diagnostic or discriminatory concentrations of Bt proteins in diet-overlay or diet-incorporation bioassays. However, the F1 screen can only be used if there is an artificially selected and maintained resistant strain which has the same Vip3A resistance allele(s) that occur in the field.

The invention is further drawn to a method of F1 screen monitoring of the frequency of the resistance alleles of the Correntina Vip-R and Tifton Vip-R strains in a fall armyworm population from a geographic location. To perform F1 screen monitoring, a plurality of fall armyworms are collected from a geographic location. The sex of each of the collected fall armyworms is determined, and breeding pairs are created with an adult of a collected fall armyworm and with an adult of the Correntina Vip-R strain or Tifton Vip-R strain. The progeny of the breeding pairs are collected and allowed to feed on a diet comprising an effective concentration of Vip3A, wherein the effective concentration is sufficient to kill susceptible fall armyworms. This may be provided as fresh corn leaf tissue comprising event MIR162. The number of surviving fall armyworms are counted and the frequency of the Vip3A-resistance allele(s) in the fall armyworm population collected from the geographic location is determined.

The following examples are intended solely to illustrate one or more preferred embodiments of the invention and are not to be construed as limiting the scope of the invention.

### EXAMPLES

### Example 1: Artificially Selected Resistant Fall Armyworm Strains

Two separate fall armyworm strains that exhibit resistance to Vip3A protein were artificially selected via laboratory breeding processes. The process by which each strain was developed is described below.

### Example 1.1: Correntina Artificially Selected Strain

The Correntina Vip3A resistant fall armyworm strain was selected using a method similar to the F2 screen method (Andow and Alstad, 1998, J Econ Entomol 91: 572-578). This method involves the collection of a large number of individuals in the field for the establishment of isofemale lines, which are formed from one male and one female. The descendants of the F1 generation from each isofemale line are then mated with each other, and the descendants of F2 are screened by a discriminatory concentration of Bt toxin, insecticide or transgenic plant expressing a Bt toxin. To practice this method, populations of about 1000 larvae were collected from commercial plantings of non-Bt corn from Correntina, Bahia, Brazil (Bernardi et al., 2015, Crop Protection, 76: 7-14, incorporated by reference herein). This corn did not express any Bt insecticidal proteins. About 550 of the collected larvae survived to the pupal stage, and of the adults 100 couples were selected as the F0 generation of the Correntina population. The descendants of the F2 generation were selected for resistance to Vip3A using either Vip3Aa20 on diet over-lay assays or excised-leaf of Agrisure® Viptera™ corn, which comprises the MIR162 event and expresses Vip3Aa20, using methods similar to that described in Benardi et al. (2005, Crop Protection, 76: 7-14, incorporated by reference herein). The concentration of Vip3Aa20 toxin used was 4000 ng/cm², which represents approximately a two-fold value of LC₉₉ for S. frugiperda populations, and is considered a diagnostic concentration (Bernardi et al., 2014, J Econ Entomol, 107: 781-790, incorporated by reference herein).

A total of 852 isofemale lines were screened in 2013 and in 2014. Only one isofemale line from Correntina, Bahia, Brazil produced larvae that survived and completed development on Agrisure® Viptera™ corn leaves that comprise the MIR162 event and express Vip3Aa20. Progenies from the offspring of this isofemale were reared on Agrisure® Viptera™ corn leaves on subsequent generations until third instar, and then transferred to artificial diet until pupation. This procedure was performed for five consecutive generations to obtain the resistant strain to Vip3Aa20, designated as Correntina Vip-R. A susceptible strain (Sus) has been maintained in the laboratory without selection pressure by insecticides for >10 yr.

### Example 1.2: Tifton Artificially Selected Strain

An initial FAW population was established in early 2012 from eggs purchased from French Agricultural Research (FAR, Minnesota). A second FAW population was collected on voluntary corn that did not express Vip3A protein from Tifton, Ga. during October 2012. After maintaining in the laboratory setting for at least 2 generations, the FAW population from Tifton, Ga., was sexed and mass reciprocally crossed with the FAW population from FAR. The progeny from the reciprocal crosses were further mass crossed. The progeny of the mass cross were considered the F1 generation. The neonates of the F2 generation were then selected for resistance to Vip3A by feeding, for seven days, an artificial diet comprising Vip3Aa19 at a concentration of 4500 ng/cm². At seven days, the larvae which had progressed to at least second instar were transferred onto diet that did not contain Vip3A. Of 1,920 neonates, 90 (5%) larvae survived and were transferred to diet in the absence of Vip3A selection. Eventually, 73 individuals successfully pupated. A mass cross was performed between adults from these Vip3A-selected larvae and the same generation adults from larvae free of Vip3A selection. Subsequent generations were raised with or without Vip3A selection, to allow for selection of Vip3A resistance but also to allow for the population to recover. Specifically, resistance selection was conducted for the following 8 generations at corresponding Vip3Aa19 concentrations: F2 generation at 4500 ng/cm², F5 and F10 generations at 415 ng/cm², F13-F16 and F24 generations at 1000 ng/cm². Here, the pupation rate of the F24 selection was 68% which is within the range of pupation rates of 50%-79% for the F13-F16 during which FAW was under the pressure of resistance selection for 4 consecutive generations. Compared to the susceptible FAR FAW population, the Vip3A selected colony could survive exposure to Vip3A at much higher concentrations. Therefore, a FAW Vip3A resistant strain, designated as Tifton Vip-R, was obtained.

### Example 2: Determination of Resistance Level for Correntina Vip-R Strain

### Example 2.1: Plant and Leaf Bioassays for Correntina Strains

To measure the phenotypic resistance to Vip3A in the Correntina Vip-R strain, whole-plant and excised-leaf bioassays were conducted with Correntina Vip-R, Sus and reciprocal crosses of Correntina Vip-R×Sus. Initially, Agrisure® Viptera™ corn (which expresses Vip3Aa20), Agrisure® Viptera™ 3 corn (which expresses Vip3Aa20 and Cry lAb) and non-Bt near-isoline (which does not express any Bt insecticidal proteins) were sowed in 12-liter plastic pots, containing soil and organic compost (1:1) and kept in a greenhouse. Four plants were maintained in each pot, totaling 100 plants (4 replicates of 25 plants per treatment). The Vip3Aa20 expression was checked using the QuickStix™ Kit for Vip3A. When plants reached the V6 stage of growth, one neonate (<24 h old) was released on each corn whorl. To prevent larval movement, plants were kept inside transparent plastic tubes (1.0 m height×0.30 m diameter), which were fixed in the pot border, and sealed at the top with a voile-type fabric and a rubber band. Pots were disposed in a completely randomized experimental design. At 10 days, surviving larvae were counted, recovered larvae were reared individually in laboratory on the corresponding corn leaves placed on a gelled mixture of agar-water at 2% in 16-well bioassay trays (Advento do Brasil, Sao Paulo, Brazil). In addition, laboratory leaf bioassays were also performed with 128 neonates per strain or cross (8 replicates of 16 larvae) individually reared in the same plants and bioassays trays described above. Leaves were changed every 48 h until pupation. Larval survival at 10 days, number of pupae and adult emergence were measured. To demonstrate that Correntina Vip-R adults obtained from larvae fed in Agrisure® Viptera™ and Agrisure® Viptera™ 3 corn produce viable offspring, 10 single pairs were mated by pairing virgin males and virgin females in PVC cages (23 cm height×10 cm diameter) lined with newsprint (oviposition substrate) and closed at the top with a voile-type fabric. The number of eggs and hatched larvae were assessed every 2 days, until the death of the female.

Plant and leaf bioassays were performed to test the hypothesis that the Correntina Vip-R strain is phenotypically resistant to the Vip3Aa20 protein expressed on corn. The first comparison was the survival of the Correntina Vip-R strain on Agrisure® Viptera™ and Agrisure® Viptera™ 3 corn tissues compared to the survival of the susceptible (Sus) strain. The second comparison was the survival of the Correntina Vip-R strain on non-Bt corn with the survival of the Sus strain in non-Bt corn. Statistical differences on larval survival at 10 days, pupae (not deformed), adult (not deformed) emergence, number of eggs and hatched larvae were determined using least-square means test (LSMEANS) at a 5% level of significance with PROC MIXED procedure in SAS 9.1.

There were significant differences among fall armyworm strains in plant bioassays for larval survival at 10 days (F=36.38; df=11, 36; P<0.0001), pupae (F=34.32; df=11, 36; P<0.0001), and adult emergence (F=49.89; df=11, 36; P<0.0001) (Table 1). Significant differences among strains was also observed in leaf bioassays for larval survival at 10 days (F=296.69; df=11, 84; P<0.0001), pupae (F=129.16; df=11, 84; P<0.0001), and adult emergence (F=83.41; df=11, 84; P<0.0001).

TABLE 1
Survival (% ± SE) from neonate to adult of fall armyworm
strains in Vip corn technologies and non-Bt.

| Strain or | | | | |
|---|---|---|---|---|
| cross | Host | Larvae^{a, b} | Pupae^{b} | Adults^{b} |
| Greenhouse | | | | |
| (plant) | | | | |
| Correntina | Agrisure ™ | 32.2 ± 6.9 b | 30.5 ± 4.6 b | 27.2 ± 6.5b |
| Vip-R | Viptera ® 3 | | | |
| | Agrisure ™ | 46.9 ± 3.2 a | 42.2 ± 6.8 a | 37.2 ± 5.9 a |
| | Viptera ® | | | |
| | Non-Bt corn | 48.3 ± 5.0 a | 45.0 ± 4.2 a | 41.7 ± 3.2 a |
| Correntina | Agrisure ™ | 0.0 ± 0.0 c | 0.0 ± 0.0 c | 0.0 ± 0.0 c |
| Vip-R♀ × | Viptera ® 3 | | | |
| Sus♂ | Agrisure ™ | 0.0 ± 0.0 c | 0.0 ± 0.0 c | 0.0 ± 0.0 c |
| | Viptera ® | | | |
| | Non-Bt corn | 42.8 ± 7.1 a | 40.1 ± 3.3 a | 36.4 ± 6.2 a |
| Correntina | Agrisure ™ | 0.0 ± 0.0 c | 0.0 ± 0.0 c | 0.0 ± 0.0 c |
| Vip-R♂ × | Viptera ® 3 | | | |
| Sus♀ | Agrisure ™ | 0.0 ± 0.0 c | 0.0 ± 0.0 c | 0.0 ± 0.0 c |
| | Viptera ® | | | |
| | Non-Bt corn | 44.6 ± 8.6 a | 42.2 ± 5.9 a | 36.0 ± 7.6 a |
| Sus | Agrisure ™ | 0.0 ± 0.0 c | 0.0 ± 0.0 c | 0.0 ± 0.0 c |
| | Viptera ® 3 | | | |
| | Agrisure ™ | 0.0 ± 0.0 c | 0.0 ± 0.0 c | 0.0 ± 0.0 c |
| | Viptera ® | | | |
| | Non-Bt corn | 44.4 ± 7.5 a | 41.4 ± 4.2 a | 40.0 ± 6.8 a |
| Laboratory | | | | |
| (leaf) | | | | |
| Correntina | Agrisure ™ | 55.5 ± 7.7 b | 39.8 ± 5.0 b | 31.3 ± 3.7 b |
| Vip-R | Viptera ® 3 | | | |
| | Agrisure ™ | 89.1 ± 1.6 a | 55.7 ± 4.5 a | 47.7 ± 5.8 a |
| | Viptera ® | | | |
| | Non-Bt corn | 91.4 ± 1.3 a | 57.8 ± 2.8 a | 55.1 ± 3.5 a |
| Correntina | Agrisure ™ | 0.0 ± 0.0 c | 0.0 ± 0.0 c | 0.0 ± 0.0 c |
| Vip-R♀ × | Viptera ® 3 | | | |
| Sus♂ | Agrisure ™ | 0.0 ± 0.0 c | 0.0 ± 0.0 c | 0.0 ± 0.0 c |
| | Viptera ® | | | |
| | Non-Bt corn | 87.8 ± 1.9a | 61.8 ± 2.7 a | 57.7 ± 4.2 a |
| Correntina | Agrisure ™ | 0.0 ± 0.0 c | 0.0 ± 0.0 c | 0.0 ± 0.0 c |
| Vip-R♂ × | Viptera ® 3 | | | |
| Sus♀ | Agrisure ™ | 0.0 ± 0.0 c | 0.0 ± 0.0 c | 0.0 ± 0.0 c |
| | Viptera ® | | | |
| | Non-Bt corn | 82.9 ± 2.7 a | 64.9 ± 5.2 a | 61.5 ± 4.8 a |
| Sus | Agrisure ™ | 0.0 ± 0.0 c | 0.0 ± 0.0 c | 0.0 ± 0.0 c |
| | Viptera ® 3 | | | |
| | Agrisure ™ | 0.0 ± 0.0 c | 0.0 ± 0.0 c | 0.0 ± 0.0 c |
| | Viptera ® | | | |
| | Non-Bt corn | 89.1 ± 1.3a | 63.0 ± 3.7 a | 59.8 ± 5.0 a |

^{a}Survival at 10 days after infestation in greenhouse and laboratory studies.
^{b}Means within a column for each study followed by the same letter are not significantly different (P > 0.05).

The Correntina Vip-R strain in plant bioassays showed significant higher survival compared to the Sus strain (larvae, F=24.96; df=3, 12; P<0.0001; pupae, F=17.76; df=3, 12; P<0.0001; adults, F=24.07; df=3, 12; P<0.0001). Similar results was observed in leaf bioassays (larvae, F=125.16; df=3, 28; P<0.0001; pupae, F=70.65; df=3, 28; P<0.0001; adults, F=47.26; df=3, 28; P<0.0001). The Correntina Vip-R strain also had similar survival compared to the Sus strain on non-Bt corn in plant (larvae, F=1.02; df=1, 6; P=0.3439; pupae, F=0.94; df=1, 6; P=0.5822; adults, F=0.80; df=1, 6; P=0.6622), and leaf bioassays (larvae, F=0.78, df=1, 14, P=0.3923; pupae, F=0.98; df=1, 14; P=0.2240; adults, F=0.52; df=1, 14; P=0.8012). In contrast, the Sus strain and reciprocal crosses presented complete mortality on tissues from both Vip3A expressing corn plants.

For true phenotypic resistance to the Vip3A protein, the surviving larvae should be able to produce viable offspring. The results here show significant differences in the number of eggs (F=2.96; df=3, 35; P=0.0454) and neonates per female (F=3.27; df=3, 35; P=0.0327) between Correntina Vip-R females from Vip corn and non-Bt corn when compared with Sus females from non-Bt corn (Table 2). In contrast, no significant differences were detected in the number of eggs (F=0.13; df=2, 26; P=0.8758) and neonates (F=0.03; df=2, 26; P=0.9740) of Correntina Vip-R females from Vip3A expressing corn plants with Correntina Vip-R females from non-Bt corn. These results showed that Correntina Vip-R strain larvae have the ability to survive on Agrisure® Viptera™ and Agrisure® Viptera™ 3 corn tissues, emerge as adults, and then produce viable offspring. This is irrefutable evidence that the Correntina Vip-R strain is phenotypically resistant to the Vip3Aa20 protein.

TABLE 2
Eggs and neonates produced by Correntina Vip-R females from
transgenic corn expressing Vip3Aa20 protein and non-Bt corn and
Sus females from non-transgenic corn

| | Development time of life stages^{a} | |
|---|---|---|
| Parameter | Eggs per female | Neonates per female |
| Agrisure ® Viptera3 ™ | 1192.25 b | 999.56 b |
| Agrisure ® Viptera ™-Vip-R | 1068.70 b | 938.40 b |
| Non-Bt corn | 1070.36 b | 966.60 b |
| Non-Bt corn-Sus | 1676.40 a | 1557.70 a |

^{a}Means within a column followed by the same letter are not significantly different for two-tailed t-tests for pairwise group comparisons (P > 0.05).

### Example 2.2: Concentration-Response in Diet-Overlay Bioassays

To perform diet-overlay bioassays we used an artificial diet commonly used for fall armyworm rearing in the laboratory (Kasten et al., 1978, Rev Agric 53: 69-78). Initially, the diet was prepared and poured on 128-well bioassay trays (BIO-BA-128, CD International Inc., Pitman, N.J., USA), at a volume of 1 ml per well. Afterwards, the Vip3Aa20 protein was diluted in distilled water to prepare the different concentrations to be tested. Surfactant Triton X-100 at 0.1% was added to obtain a uniform spread of the solution over the diet surface. The control treatment was composed by distilled water and surfactant. Six to eight concentrations were used to exposed the Correntina Vip-R strain (1,120 to 200,000 ng/cm²), and separately the Sus and reciprocal crosses (20 to 640 ng/cm²) to the Vip3A protein. These concentrations were applied on the diet surface. The surface area in each well was 1.5 cm². After a drying period, one neonate (<24 h old) was placed to each well (at minimum of 64 neonates were tested per concentration). The trays were sealed with self-adhesive plastic sheets (BIO-CV-16, CD International Inc.), and then placed in a climate chamber at 27±1° C., 60±10% relative humidity, and a photoperiod of 14:10 (L:D) h. Mortality and growth inhibition response was measured at seven days.

The concentrations used to estimate LC₅₀ and EC₅₀ values showed a similar range (Tables 3 and 4). The LC₅₀ and EC₅₀ could not be accurately estimated for the Correntina Vip-R strain, due to lack of concentration-response at highest concentrations. The mortality-response of Correntina Vip-R strain at the maximum concentration of 200,000 ng/cm² did not exceed 40%. In contrast, Sus and reciprocal crosses showed complete mortality at 1120 ng/cm².

TABLE 3
Mortality of fall armyworm strains to Vip3A insecticidal protein
Mortality response

| | | | Correntina | | Correntina | | |
|---|---|---|---|---|---|---|---|
| | Sus | | Vip-R♀ × Sus♂ | | Vip-R♂ × Sus♀ | | Correntina Vip-R |
| Conc. ng/cm² | Estim. | Observ | Estim. | Observ. | Estim. | Observ. | Estim. |
| 0 | | | | | | | |
| 20 | 3.9 | 3.94 | 3.89 | 4.01 | 3.82 | 3.88 | |
| 36 | 4.47 | 4.36 | 4.35 | 4.55 | 4.28 | 4.37 | |
| 74 | 5.03 | 4.85 | 4.81 | 4.85 | 4.72 | 4.61 | |
| 112 | 5.57 | 5.91 | 5.25 | 4.93 | 5.16 | 5.16 | |
| 200 | 6.13 | 6.29 | 5.71 | 5.58 | 5.61 | 5.47 | |
| 360 | 6.70 | 6.39 | 6.17 | 6.39 | 6.07 | 6.28 | |
| 640 | 7.26 | 7.13 | 6.63 | 7.13 | 6.51 | 6.38 | |
| 2000 | | | | | | | 3.77 |
| 3600 | | | | | | | 3.85 |
| 6400 | | | | | | | 3.94 |
| 11200 | | | | | | | 4.02 |
| 20000 | | | | | | | 4.10 |
| 36000 | | | | | | | 4.18 |
| 64000 | | | | | | | 4.27 |
| 112000 | | | | | | | 4.35 |
| 200000 | | | | | | | 4.43 |
| Intercept | −4.00 | | −3.48 | | −3.51 | | −2.23 |
| Slope | 2.23 | 0.21 | 1.82 | 0.19 | 1.79 | 0.17 | 0.33 |

TABLE 4
Growth inhibition response of fall armyworm
strains to Vip3A insecticidal protein
Growth inhibition

| | | Correntina Vip- | Correntina Vip- | Correntina |
|---|---|---|---|---|
| | Sus | R ♀ × Sus♂ | R♂ × Sus♀ | Vip-R |
| Conc. ng/cm² | % | % | % | % |
| 0 | 0.00 | 0.00 | 0.00 | 0.00 |
| 20 | 20.30 | 26.77 | 32.12 | |
| 36 | 41.00 | 40.89 | 36.18 | |
| 64 | 53.00 | 44.67 | 46.77 | 22.62 |
| 112 | 67.75 | 63.51 | 55.95 | 32.10 |
| 200 | 85.51 | 77.48 | 82.37 | 31.72 |
| 360 | 89.48 | 86.08 | 91.60 | 29.92 |
| 640 | 99.39 | 97.71 | 96.00 | 33.54 |
| 1120 | 100.00 | 100.00 | 100.00 | 29.66 |
| 2000 | | | | 32.95 |
| 3600 | | | | 34.23 |
| 6400 | | | | 38.07 |
| 11200 | | | | 37.48 |
| 20000 | | | | 37.53 |
| 36000 | | | | 25.59 |
| 64000 | | | | 23.28 |
| 112000 | | | | 17.45 |
| 200000 | | | | 25.17 |

### Example 3: Genetic Characterization of Vip3A Resistance for Correntina Vip-R Strain

### Example 3.1: Inheritance of Vip3A Resistance

To evaluated the inheritance of resistance reciprocal crosses were performed between the Correntina Vip-R and Sus strains with at least 40 pairs per cross. Neonate larvae of the Correntina Vip-R, Sus and reciprocal crosses were tested for susceptibility to Vip3A protein by using the diet-overlay bioassays with seven concentrations of Vip3Aa20 ranging from 1120 to 200,000 ng/cm² for the Correntina Vip-R strain and from 20 to 1,120 ng/cm² for Sus and reciprocal crosses. Diet-overlay bioassays were performed as described in Example 2.2.

The concentration-mortality data in diet-overlay bioassay were submitted to Probit analysis to estimate the LC₅₀ (lethal concentration that kills 50% of larvae) and respective confidence interval (95% CI) using PROC PROBIT procedure in SAS 9.1 (SAS, Cary, N.C.). Weight data were analyzed with nonlinear regression to estimate the EC₅₀ (effective concentration that reduces weight gain by 50%) and respective confidence interval (95% CI) in JMP 9 software (SAS, Cary, N.C.). LC₅₀ and EC₅₀ were considered significantly different among treatments when their 95% CI did not overlap. Resistance Ratios (RR) were estimated by dividing LC₅₀ or EC₅₀ of Correntina Vip-R strain or reciprocal crosses by LC₅₀ or EC₅₀ of the Sus strain. In addition, the 95% CI of RR based on LC₅₀ was also estimated. To verify the effective dominance (D_{ML}) the survival of the Correntina Vip-R strain was measured relative to the Sus and reciprocal crosses.

For the Correntina Vip-R strain it was not possible to estimate the slope, LC₅₀ and 95% confidence interval of EC₅₀, but they were much higher than those estimated for the Sus or the reciprocal crosses (Tables 5 and 6). The slopes, LC₅₀ and EC₅₀ in the reciprocal crosses were not statistically different from each other based on the overlapping 95% CI. In contrast, the slope of the Sus strain was significant different from the reciprocal crosses. The resistance ratio of the Correntina Vip-R strain based on LC₅₀ and EC₅₀ was >3200 and 3000-fold, respectively. In contrast, reciprocal crosses had similar mortality and growth inhibition response of the Sus strain.

TABLE 5
Concentration-mortality (LC) response of Spodoptera frugiperda strains to the
Vip3A protein.

| | | Mortality | | | | |
|---|---|---|---|---|---|---|
| | | | Slope | LC₅₀^{a} | | RR^{c} |
| Strain or cross | Generation | n | (±SE) | (95% CI) | χ² (df)^{b} | (95% CI) |
| Correntina Vip-R | F₆ | 896 | — | >200,000.00 | — | >3,208.72 |
| Correntina Vip-R♀ × Sus♂ | F₇ | 512 | 1.82 | 90.9 | 3.90 (5) | 1.46 |
| | | | (±0.19) | (68.82- | | (0.88- |
| | | | | 112.87) | | 2.06) |
| Correntina Vip-R♂ × Sus♀ | F₇ | 576 | 1.79 | 101.62 | 3.72 (6) | 1.63 |
| | | | (±0.17) | (73.98- | | (0.95- |
| | | | | 118.93) | | 2.20) |
| Correntina Vip-R × Sus Pooled | F₇ | 1088 | 1.84 | 86.44 | 7.66 (6) | 1.38 |
| | | | (±0.12 ) | (71.93- | | (0.72- |
| | | | | 101.66) | | 1.92) |
| Sus | — | 578 | 2.23 | 62.33 | 7.27 (5) | 1 |
| | | | (±0.21) | (50.67- | | |
| | | | | 74.60) | | |

^{a}LC₅₀: concentration of Vip3Aa20 (ng/cm²) required to kill 50% of larvae at 7 days.
^{b}P > 0.05 in the goodness-of-fit test.
^{c}Resistance Ratio (RR) = LC₅₀ of Correntina Vip-R or reciprocal crosses/LC₅₀ of Sus strain.

TABLE 6
Growth inhibition (EC) response of Spodoptera frugiperda
strains to the Vip3A protein

| | | Growth inhibition | | |
|---|---|---|---|---|
| | Gener- | | EC₅₀^{d} | |
| Strain or cross | ation | n | (95% CI) | RR^{c} |
| Correntina Vip-R | F₆ | 410 | 153,990.00 | 3,066.30 |
| | | | (not calculated)^{e} | |
| Correntina Vip-R♀ × | F₇ | 237 | 62.39 | 1.24 |
| Sus♂ | | | (27.85-100.91) | |
| Correntina Vip-R♂ × | F₇ | 267 | 64.6 | 1.29 |
| Sus♀ | | | (38.28-90.92) | |
| Correntina Vip-R × Sus | F₇ | 504 | 60.98 | 1.21 |
| Pooled | | | (29.04-97.80) | |
| Sus | — | 211 | 50.22 | 1 |
| | | | (24.16-75.83) | |

^{c}Resistance Ratio (RR) = EC₅₀ of Correntina Vip-R or reciprocal crosses/EC₅₀ of Sus strain.
^{d}EC₅₀: effective concentration of Vip3Aa20 (ng/cm²) required to cause 50% growth inhibition at 7 days.
^{e}not calculated due to insufficient dose response.

### Example 3.2: Effective Dominance of Vip3A Resistance

Effective dominance was estimated for the Correntina Vip-R strain. Reciprocal crosses were conducted between the Correntina Vip-R and Sus strains. In addition, Correntina Vip-R and Sus intra-population crosses were also performed. Neonate larvae from each cross were tested individually in four 128-well bioassays trays; 64 wells per tray with Vip3Aa20 protein at 2,000 or 3,600 ng/cm² and 64 wells without insecticidal protein. This concentration has been used for fall armyworm resistance monitoring in Brazil (Bernardi et al., 2014, J Econ Entomol, 107: 781-790). Diet-overlay bioassays were performed as described in Example 2.2.

No larvae survived from Sus and the reciprocal crosses when exposed to 2,000 and 3,600 ng/cm² of Vip3Aa20 protein (Table 7). Neonates from the Correntina Vip-R strain when exposed in these two concentrations survived at similar rate to the controls. The larval survival in controls ranged from 93 to 97%. Dominance was calculated as D_{ML}=0.0±0.0 for the Correntina Vip-R strain, and confirmed to be completely recessive at 2,000 and 3,600 ng/cm² of Vip3Aa20 protein.

TABLE 7
Survival (% ± SE) of Spodoptera frugiperda strains at diagnostic
concentrations of Vip3A protein.

| | | | Dominance |
|---|---|---|---|
| Cross | Survival at 7 days (%) | | (D_{ML})^{a} |
| | 2000 ng/cm² Vip3Aa20 | Without Vip3Aa20 | |
| Correntina Vip- | 93.7 ± 1.9 | 95.3 ± 1.5 | 0.0 ± 0.0 |
| R♀ × | | | |
| Correntina Vip- | | | |
| R♂ | | | |
| Correntina Vip- | 0.0 ± 0.0 | 95.7 ± 1.0 | |
| R♀ × Sus♂ | | | |
| Correntina Vip- | 0.0 ± 0.0 | 94.6 ± 0.9 | |
| R♂ × Sus♀ | | | |
| Sus ♂ × Sus♀ | 0.0 ± 0.0 | 94.9 ± 1.1 | — |
| | 3600 ng/cm² Vip3Aa20 | Without Vip3Aa20 | |
| Correntina Vip- | 92.6 ± 1.9 | 97.3 ± 0.8 | 0.0 ± 0.0 |
| R♀ × | | | |
| Correntina Vip- | | | |
| R♂ | | | |
| Correntina Vip- | 0.0 ± 0.0 | 93.4 ± 1.3 | |
| R♀ × Sus♂ | | | |
| Correntina Vip- | 0.0 ± 0.0 | 93.0 ± 1.8 | |
| R♂ × Sus♀ | | | |
| Sus♂ × Sus♀ | 0.0 ± 0.0 | 94.1 ± 1.4 | — |

^{a}Effective dominance at two concentrations of Vip3Aa20 protein used for the resistance monitoring of S. frugiperda in Brazil.

### Example 3.3: Number of Genes Associated with Vip3A Resistance

To estimate the number of genes influencing resistance, F₁ progeny from reciprocal cross (Correntina Vip-R♀×Sus♂) were backcrossed with the parental phenotypically more distinct, in this case, the Correntina Vip-R strain (c and 2). Neonates from backcrosses were exposed to leaf-discs of Agrisure® Viptera™ and Agrisure® Viptera™ 3 corn placed on 12-well bioassay trays (Corning, Tewksbury, Mass., USA) containing gelled mixture of water-agar 2%. Leaf discs were separated from the water-agar layer by a filter paper disc. Then, one neonate was placed on each well (120 neonates tested per backcross). Trays were sealed with a plastic film, and placed in a climatic chamber at 27±1° C., 60±10% relative humidity, and a photoperiod of 14:10 (L:D) h. Mortality was measured at four days.

To estimate the number of genes affecting resistance, the observed and expected mortality of F₁ progeny from backcrosses on leaf-discs of Agrisure® Viptera™ and Agrisure® Viptera™ 3 were submitted to the chi-squared test. The hypothesis of monogenic inheritance is rejected if the calculated chi-squared ≥tabulated chi-squared with 1 degree of freedom.

The fitness cost components was submitted to the same statistical procedure described for plant and leaf bioassays. The putative deviation in the sex ratio was analyzed using the chi-square test with PROC FREQ procedure in SAS 9.1. A life table was also calculated by estimating the mean generation time (7), the net reproductive rate (Rₒ), the intrinsic rate of increase (rₘ) and the finite rate of increase (λ). The life table parameters were estimated using “lifetable.sas” procedure in SAS 9.1.

There were no significant deviation between observed and expected mortality in the backcrosses when exposed to leaf-disc of Agrisure™ Viptera® 3 and Agrisure™ Viptera® corn (Table 8). These results suggest that resistance was controlled by a single major gene in the resistant strain. In addition, we used Lande's method (Lande et al., 1981, Genetics, 99: 541-533; Tabashnik et al., 1992, J Econ Entomol, 84: 1046-1055) to estimate the minimum number of independently segregating genes. According to this method, Correntina Vip-R strain showed that a small number of loci affected the resistance to Vip3A protein. It was estimated that the minimum number of independently segregating loci controlling resistance was <1. This indicates that only one or a few genes are involved with resistance.

TABLE 8
Direct test for deviation between observed and expected mortality for a
monogenic model (df = 1).

| | Mortality on leaf-disc at 4 days (%) | | | | | | | |
|---|---|---|---|---|---|---|---|---|
| | Agrisure ™ Viptera ® 3 | | | | Agrisure ™ Viptera® | | | |
| Backcross | Observed | Expected | χ² | P | Observed | Expected | χ² | P |
| F1♂ × | 66.9 | 55.5 | 2.62 | 0.13^{a} | 64.2 | 52.9 | 2.52 | 0.12^{a} |
| Correntina | | | | | | | | |
| Vip-R♀ | | | | | | | | |
| F1♀ × | 68.4 | 55.0 | 3.56 | 0.07^{a} | 65.0 | 52.5 | 2.98 | 0.08^{a} |
| Correntina | | | | | | | | |
| Vip-R♂ | | | | | | | | |

^{a}Probability values indicating no significant differences between the observed and expected mortality (P < 0.05).

### Example 4: Fitness Costs Associated with Vip3A Resistance for the Correntina Vip-R Strain

To evaluate the presence of fitness costs associated with Vip3A resistance in the Correntina Vip-R strain, laboratory studies using non-Bt near-isoline corn leaves were performed for the Correntina Vip-R and Sus strains and reciprocal crosses. Bioassays were started when plants reached the V6 stage of growth. Leaves were removed from the corn whorl, cut into pieces and placed on 16-well bioassay trays (Advento do Brasil, Sao Paulo, Brazil) containing gelled mixture of agar-water at 2%. Leaves were separated from the agar-water layer by a filter paper disc. For each strain or cross, one neonate (<24 h old) of F7 generation was placed into each well. The bioassays were conducted in a climate chamber at 27±1° C., 60±10% relative humidity and a photoperiod of 14:10 (L:D) h. Leaves were changed every 48 h over the larval development period. The experimental design was completely randomized with 10 replicates (16 larvae per replicate) per strain or cross. The following fitness cost components were measured: development time and survival rate of egg, larvae, pupae and egg to adult period; sex ratio; female longevity; timing of pre-oviposition, oviposition and post-oviposition and fecundity (total eggs per female). Development time and survival rate of egg, larvae, pupae and egg-to-adult period were determined in daily observations. Female longevity and fecundity were evaluated in 20 pairs per strain or cross that were kept in PVC cages (23 cm height×10 cm diameter) lined with newsprint (oviposition substrate) and closed at the top with a voile-type fabric. The number of eggs and female mortality were assessed daily. To evaluate the embryonic period and survival, 100 eggs were obtained from the second oviposition of each pair. Eggs were placed into glass tubes with flat bottoms (8.5×2.5 cm). A piece of filter paper (2×1 cm) moistened with distilled water was added to the tube and closed at the top with plastic film. The number of eggs and hatched larvae were counted daily.

There was no significant differences in the development time of egg (F=1.10; df=3, 36; P=0.5830), pupae (F=2.47; df=3, 36; P=0.0538) and egg-to-adult (F=1.42; df=3, 36; P=0.2518) life stages of fall armyworm strains on non-Bt corn (Tables 9 and 10). However, the larvae stage of the Correntina Vip-R strain and of reciprocal crosses was significantly longer compared to the Sus strain (F=13.04; df=3, 36; P<0.0001). No significant differences among fall armyworm strains was also observed in the survival rate of egg (F=1.97; df=3, 36; P=0.2832) and pupae (F=2.44; df=3, 36; P=0.0800) stages. In contrast, larval survival of the Correntina Vip-R strain was significantly lower (F=5.12; df=3, 36; P=0.0047) (Table 10). This negatively affected the number of Correntina Vip-R insects that completed the life cycle, with 60% of the insects reaching the adult stage (F=8.30; df=3, 36; P=0.0003). For the Sus strain and reciprocal crosses more than 70% of insects originated adults.

TABLE 9
Development time of life stages of fall
armyworm strains fed on non-Bt corn.

| | Development time of life stages^{a} | | | |
|---|---|---|---|---|
| Parameter | Egg | Larvae | Pupae | Egg-Adult |
| Correntina Vip-R | 3.00 a | 16.41 a | 7.91 a | 27.20 a |
| Correntina Vip-R♀ × | 2.90 a | 16.26 a | 7.83 a | 26.76 a |
| Sus♂ | | | | |
| Correntina Vip-R♂ × | 3.20 a | 16.26 a | 8.07 a | 27.27 a |
| Sus♀ | | | | |
| Sus | 3.30 a | 15.18 b | 8.58 a | 26.41 a |

^{a}Means within a column followed by the same letter are not significantly different for two-tailed t-tests for pairwise group comparisons (P > 0.05).

TABLE 10
Survival rates of life stages of fall armyworm strains fed on non-Bt corn.

| | Survival rates of life stages^{a} | | | |
|---|---|---|---|---|
| Parameter | Egg | Larvae | Pupae | Egg-Adult |
| Correntina Vip-R | 91.00 a | 77.00 b | 88.00 a | 61.00 a |
| Correntina Vip-R ♀ × Sus♂ | 94.00 a | 83.00 a | 95.00 a | 69.00 a |
| Correntina Vip-R ♂ × Sus♀ | 92.00 a | 84.00 a | 92.00 a | 71.00 a |
| Sus | 91.00 a | 85.00 a | 94.00 a | 73.00 a |

^{a}Means within a column followed by the same letter are not significantly different for two-tailed t-tests for pairwise group comparisons (P > 0.05).

The sex ratio was similar among fall armyworm strains, ranging from 0.51 to 0.53 (χ²=12.92; df=3, 430; P=0.4578). In addition, biological parameters of females as longevity (F=1.63; df=3, 64; P=0.1920), timing of pre-oviposition (F=2.21; df=3, 59; P=0.0533) and post-oviposition (F=1.76; df=3, 59; P=0.1424) showed no significant differences (Table 11). In contrast, the timing of oviposition was significantly lower for reciprocal crosses and the Correntina Vip-R strain (F=3.34; df=3, 59; P=0.0253). However, only Correntina Vip-R females presented significant reduction in the number of eggs (F=12.63; df=3, 59; P<0.0001) (Table 11).

TABLE 11
Biological parameters of females of Spodoptera frugiperda
strains obtained from larvae fed on non-Bt corn.

| | Biological parameters of females^{a} | | | | |
|---|---|---|---|---|---|
| Female | Pre- | | Post- | Female | Eggs per |
| parameter | oviposition | Oviposition | oviposition | longevity | Famale |
| Correntina | 5.19 a | 5.19 a | 3.13 a | 13.11 a | 979.41 b |
| Vip-R | | | | | |
| Correntina | 4.75 a | 4.40 a | 3.18 a | 13.00 a | 1360.55 a |
| Vip-R ♀ × | | | | | |
| Sus♂ | | | | | |
| Correntina | 4.63 a | 5.21 a | 2.95 a | 12.09 a | 1310.11 a |
| Vip-R ♂ × | | | | | |
| Sus♀ | | | | | |
| Sus | 4.38 a | 6.69 b | 3.15 a | 11.69 a | 1445.31 a |

^{a}Means within a column followed by the same letter are not significantly different for two-tailed t-tests for pairwise group comparisons (P > 0.05).

All life table parameters of the Correntina Vip-R strain differ compared to reciprocal crosses and Sus strain (Table 12). The Correntina Vip-R strain showed a significant increase in the mean generation time (7) (by 2 days) and reduction of 50% in the net reproductive rate (Rₒ). Based on this, it is estimated that after 34 days (7) of Correntina Vip-R strain development in non-Bt corn, 312 females are expected to result from each female, whereas on the reciprocal crosses and Sus strain 600 females are expected after 32 days. Furthermore, the development of the Correntina Vip-R strain in non-Bt corn reduced the intrinsic (rₘ) and finite (λ) rate of increase by 15 and 2.5%, respectively. These results show the presence of relevant fitness cost associated with the Correntina Vip-R strain, but lack of fitness costs in heterozygous insects. The presence of relevant fitness cost associated with Vip3A resistance is a positive aspect for resistance management. This fitness would predict that removal of the selective agent from the environment would result in reduced resistance frequency.

TABLE 12
Population growth parameters of
fall armyworm strains fed in non-Bt corn.

| Strain | Population growth parameter^{a, b} | | | |
|---|---|---|---|---|
| or cross | T | Rₒ | rₘ | λ |
| Correntina | 34.5 ± 0.2 b | 265.5 ± 43.2 b | 0.16 ± 0.005 b | 1.18 ± 0.006 b |
| Vip-R | | | | |
| Correntina | 32.6 ± 0.3 a | 532.5 ± 68.2 a | 0.19 ± 0.004 a | 1.21 ± 0.005 a |
| Vip-R♀ × | | | | |
| Sus♂ | | | | |
| Correntina | 32.8 ± 0.3 a | 489.5 ± 52.8 a | 0.19 ± 0.004 a | 1.21 ± 0.005 a |
| Vip-R♂ × | | | | |
| Sus♀ | | | | |
| Sus | 32.2 ± 0.2 a | 530.2 ± 69.1 a | 0.19 ± 0.005 a | 1.22 ± 0.005 a |

^{a}T = mean length of a generation (days); Rₒ = net reproductive rate (females per female per generation); rₘ = intrinsic rate of population increase (per day) and λ = finite rate of population increase (per day).
^{b}Means within a column followed by the same letter are not significantly different for two-tailed t-tests for pairwise group comparisons (P > 0.05).

### Example 5: Ft Screen Monitoring of Geographic Locations for Vip3A-Resistant Fall Armyworms

The data shown in these examples demonstrate that the in planta Vip3Aa20 expression should be capable of killing heterozygous (individuals carrying a copy of resistance allele and a copy of the susceptible allele) insects, so that the resistance trait is phenotypically or ‘functionally’ recessive, which is expected to slow the rate of resistance evolution. For the success of current Vip3A resistance management strategies, it is necessary to continuously monitor for the frequency of Vip3A resistance in field populations of fall armyworms, include the presence of the resistance allele in a heterozygous insect. To do these, there are three primary methods: molecular marker analysis, the F2 screen, and the F1 screen. The F1 screen is more practical and faster than the F2 screen, and most efficient to estimate the frequency of resistance compared to phenotypic monitoring methods that use diagnostic or discriminatory concentrations of Bt proteins in diet-overlay or diet-incorporation bioassays. However, the F1 screen can only be used if there is an artificially selected and maintained resistant strain which has the same Vip3A resistance allele(s) that occur in the field.

To validate F1 screen monitoring methodology, fall armyworms were collected from nine geographic locations. The sex of each of the collected fall armyworms was determined, and breeding pairs were created with an adult of a collected fall armyworm and with an adult of an artificially selected Correntina Vip-R strain. 128 F1 progeny larvae each from a total of 263 family lines were collected and allowed to feed on Agrisure® Viptera™ or Agrisure® Viptera™ 3 fresh leaf tissue. The number of surviving fall armyworms were counted and the frequency of the Vip3A-resistance allele(s) in the fall armyworm population collected from each of the geographic locations was determined. To estimate the frequency of the Vip3Aa20 resistance allele, Equation (4) as proposed by Yue et al. (2008, Entomologia Experimentalis et Applicata 129: 172-180) was used. The confidence interval (95% CI) was estimated from equation (15) reported by Andow and Alstad (1999). The probability of false negative in F1 screen, calculated from the mortality in the control, the number of tested insects and number (unknown) of resistant insects in for each family was estimated by equation (5) reported by Yue et al. (2008). The frequency of the resistance allele and the confidence intervals were calculated using the binom.bayes function binom package R 3.1.0 (Team 2014). Although the sample size is too small to accurately determine the frequency of R alleles in each population, the F1 screen methodology was validated as being efficient for detection of FAW larvae that carry Vip3A-resistant allele(s).

### Example 6: Determination of Resistance Level for Tifton Vip-R Strain

### Example 6.1: Bioassays for Tifton Vip-R Strain

Resistance level is commonly determined by comparing the LC₅₀ values between a resistant population and a relevant susceptible population. The LC₅₀ was determined by dose response bioassays in 24-well plate format in which neonates were exposed to diet with the surface being coated with Vip3A protein at defined concentrations and one insect was used for each well. Mortality was examined after 7 days of exposure. An insect was defined dead if it was sluggish compared to normal first instar larvae in addition to its failure to advance to the second instar. The bioassay data were analyzed using a Probit program.

For the Tifton Vip-R strain, it was infeasible to obtain the LC₅₀ value because the Tifton Vip-R strain could survive exposure to Vip3A at high concentrations. When Tifton Vip-R neonates were exposed to Vip3A at 100 and 200 μg/cm² in three replicates with at least 12 neonates being exposed for each concentration in each replication, larva mortalities (2.8% for 100 μg/cm² and 0% for 200 μg/cm²) were consistently low and comparable to the control mortality (4.2%). This result suggests that the Vip3A LC₅₀ value for the Tifton Vip-R strain is much higher than 200 μg/cm².

In contrast, the susceptible FAW neonates were very sensitive to Vip3A. A bioassay performed in quadruplicate, with 24 neonates per assay at a Vip3A concentration of 150 ng/cm² consistently found that all of the FAW neonates died. For these experiments, the LC₅₀ value was 20.3 ng/cm² with 95% confidence interval (CI) between 16.4-24.1 ng/cm² and the slope for the log concentration probit curve was 2.3 with 95% CI between 2.0-2.7. Given that Vip3A LC₅₀ value for the Tifton Vip-R strain is much higher than 200 μg/cm², we can conclude with high confidence that the resistance level in the Tifton Vip-R strain should be much higher than 9852-fold.

### Example 6.2: Resistance Level Stability for Tifton Vip-R Strain

It is well known that insecticide resistance could have fitness cost and one manifestation of fitness cost is the reversal of resistance level when selection pressure no longer exists. The resistance of the Tifton Vip-R strain to Vip3A seems fairly stable as indicated by two parameters, both of which were relatively stable. One parameter is the pupation rate (neonates surviving to pupa stage) of the Tifton Vip-R strain. After 7 consecutive generations of being maintained without Vip3A in the diet (generations F17-F23), larvae from the F24 generation of the Tifton Vip-R strain was fed a diet comprising Vip3A at a concentration of 1000 ng/cm². The pupation rate of this generation was 68%. This pupation rate is comparable to the pupation rate observed when the Tifton Vip-R strain was maintained for four consecutive generations with Vip3A selection pressure, which was 50%-79%. The second parameter is the mortality rate of the Tifton Vip-R neonates when maintained on diet with or without Vip3A when exposed to Vip3A in a subsequent generation. After three consecutive generations being maintained without Vip3A in the diet (F17-F19), a subset of the Tifton Vip-R neonates of the F20 generation were fed a diet comprising Vip3A at a concentration of 200 μg/cm². The mortality rate was about 8%. Following four additional consecutive generations of being maintained without Vip3A selection (F20-23), the Tifton Vip-R neonates of the F24 generation were fed a diet comprising Vip3A at a concentration of 1000 ng/cm². The progeny of the F24 generation were maintained without Vip3A selection, and then a subset of the Tifton Vip-R neonates of the F26 generation were fed a diet comprising Vip3A at a concentration of 200 μg/cm². The mortality rate was about 0%. Both of mortality rates of Tifton Vip-R neonates from the F20 and F26 generations are well below 50% mortality, indicating that resistance level did not change dramatically.

### Example 7: Genetic Characterization of Vip3A Resistance for Tifton Vip-R Strain

Knowledge on basic genetics of resistance is important in providing insight for resistance management. For example, refuge strategy is much more effective when the resistance trait is inherited as recessive than as dominant. To determine resistance inheritance in the Tifton Vip-R strain, mass reciprocal crosses between the Vip3A susceptible colony and the Tifton Vip-R strain are performed. F1 neonates from both reciprocal crosses ((F1 (♂Sus×♀ Tifton-R), F1 (♂Tifton-R×♀ Sus)) are subject to dose response bioassays side by side along with both Sus and Tifton-R strains. By comparison of the dose-response curves among the susceptible population, the two F1 progenies and the resistant Tifton-R strain, we can determine the resistance inheritance in the Tifton Vip-R strain. Differences in dose response curves between the two F1 progenies would suggest maternal contribution to the resistance. If the curves for both F1 progenies are very close to that of the susceptible colony, the resistant trait in the Tifton Vip-R strain is likely inherited as a recessive trait. Similarly, if the curves for both F1 progenies are very close to that of the resistant population, the resistant trait in the Tifton Vip-R strain is likely inherited as a dominant trait. If the curves for both F1 progenies sit somewhere in between, the resistance is likely inherited as an incompletely dominant or incompletely recessive trait.

### Example 8: Fitness Costs Associated with Vip3A Resistance for the Tifton Vip-R Strain

Generally two approaches are used to measure fitness costs. One approach measures fitness components that can be development time, growth rate, body mass, survival, etc. and the other approach measures fitness holistically by covering costs across all fitness components. To determine the fitness costs associated with Vip3A resistance for the Tifton Vip-R strain, neonates from the Tifton Vip-R strain and the susceptible FAW colony are maintained on a diet that does not include Vip3A protein. Throughout their lifecycles, development time, growth rate, body mass and pupation rate are measured.

Although the foregoing invention has been described in some detail by way of illustration and example for purposes of clarity and understanding, it will be clear to those of skill in the art that certain changes and modifications may be practiced within the scope of the appended claims.

What is claimed:

1. A method of evaluating the activity of a compound on a Vip3A-resistant fall armyworm, comprising:
a) exposing one or more Vip3A-resistant fall armyworms to a compound; and
b) determining the activity of the compound on the one or more Vip3A-resistant fall armyworms;
to thereby evaluate if said compound is active on a Vip3A-resistant fall armyworm.

2. The method of claim 1 further comprising selecting a compound for further development when said compound exhibits toxicity to said Vip3A-resistant fall armyworm.

3. The method of claim 1, wherein the compound is an insecticidal protein.

4. A method for producing a field-derived, artificially selected strain of Vip3A-resistant fall armyworms, the method comprising:
a) collecting a plurality of fall armyworms from a geographic location;
b) allowing the fall armyworms to feed on a diet comprising an effective concentration of Vip3A, sufficient to kill susceptible fall armyworms;
c) selecting one or more of the surviving fall armyworms from step (b);
d) forming a strain of surviving artificially selected fall armyworms that comprise field-derived resistance to Vip3A.

5. The method of claim 4, wherein the geographic location is in or proximal to an agricultural field.

6. The method of claim 5, wherein the agricultural field comprises corn plants.

7. The method of claim 6, wherein the corn plants express a Vip3A protein.

8. The method of claim 7, wherein the Vip3A protein is Vip3Aa20.

9. A method for producing an artificially selected strain of Vip3A-resistant fall armyworms, the method comprising:
a) collecting a plurality of fall armyworms from a geographic location, wherein said plurality of fall armyworms constitute the F0 generation;
b) allowing the field-collected fall armyworms to breed unselectively for one generation, thereby producing an F1 generation which has not been feed a diet comprising a Vip3A protein;
c) determining the sex of the F1 generation adults;
d) selecting for breeding pairs of the F1 generation and allowing the breeding pairs to mate, thereby producing an F2 generation;
e) allowing fall armyworm larvae of the F2 generation to feed on a diet comprising an effective concentration of Vip3A, sufficient to kill susceptible fall armyworms;
f) selecting one or more surviving fall armyworms from step (e); and
g) forming an artificially selected strain of surviving fall armyworms that comprise resistance to Vip3A.

10. The method of claim 9, wherein the geographic location is in or proximal to an agricultural field.

11. The method of claim 10, wherein the agricultural field comprises corn plants.

12. The method of claim 4, further comprising:
e) mating a Vip3A-resistant fall armyworm produced by the method with a fall armyworm that is susceptible to Vip3A, whereby progeny larvae are produced;
allowing the larvae resulting from step (e) to feed on a diet comprising an effective concentration of Vip3A, sufficient to kill susceptible fall armyworms; and
g) analyzing the mortality rates of the progeny from each mating.

13. The method of claim 12, further comprising backcrossing progeny larvae of step e) with the Vip3A-resistant fall armyworms to produce a backcrossed progeny.

14. The method of claim 12 wherein the method further comprises determining the inheritance of resistance of the strain of Vip3A-resistant fall armyworms.

15. The method of claim 4, wherein the diet comprising an effective concentration of Vip3A comprises leaf material from maize plants comprising event MIR162.
